(19) United States
(12) Plant Patent Application Publication
(10) Pub. No.: US 2010/0199395 P1
(43) Pub. Date: Aug. 5, 2010

(54) Brugmansia Angel's Summer Dream

(50) ;

(76) Inventor: Byron E. Martin, Danielson, CT (US)

Correspondence Address: GREENBERG & LIEBERMAN, LLC, 2141 WISCONSIN AVE, N.W., SUITE C-2, WASHINGTON, DC, 20007, US

(21) Appl. No.: 12/322,675

(22) Filed: Feb. 5, 2009

(51) Int. Cl. A01H 5/00

(52) U.S. Cl. PLT/216

(57) ABSTRACT

A new cultivar of the Brugmansia genus which exhibits lateral branching, enabling specimens to flower between at a height between twelve and twenty-four inches, longer duration of flowering, early bloom iniations on young plants and is a compact, free flowering producer of non-white colored blooms.

### BACKGROUND OF THE INVENTION

The present invention relates to a new and distinct cultivar from the Brugmansia genus. Botanically, the present invention is known as Brugmansia hybrida, hereinafter referred to by its common name, Angel's Summer Dream.

The Brugmansia genus consists of six species of plants that are native to subtropical regions of South America. Typical specimens of this genus reach between three and eleven meters and exhibit large, pendulous, trumpet-shaped flowers. Brugmansias have tan, slightly rough bark and exhibit large, alternate leaves with a coarsely toothed margin and which are covered in fine hairs.

The present invention is the product of a meticulously planned breeding program designed and conducted by the Inventor in Danielson, Conn. in the summer of 2007. The Inventor set about this project with several objectives: to create a Brugmansia that would exhibit a longer duration of flowering, earlier bloom initiation on young plants and, furthermore, the Inventor desired to breed a compact, free-flowering Brugmansia that did not produce a white-hued flower. The present invention is the successful product of this breeding program as it exhibits all of the aforementioned traits and characteristics including producing not a white flower but rather a flower exhibits blooms ranging from pale-orange to golden-yellow, depending on the environmental conditions surrounding any given specimen of Angel's Summer Dream. Additionally, Angel's Summer Dream exhibits a lateral branching pattern that enables the plant to grow more laterally than it grows vertically. As such the plant is able to bloom when it reaches a height between twelve and twenty-four inches, a vast difference from previous varieties of the Brugmansia genus which require a height between three and five feet in order to bloom.

This new Brugmansia stems from a cross-pollination enacted by the Inventor wherein Brugmansia Cherub was utilized as the female, or pod, parent and Brugmansia Inca Sun was used as the male, or seed, parent.

### SUMMARY OF THE INVENTION

The cultivar Angel's Summer Dream is a tropical plant that thrives most fully in warm climates. As such, it has not been observed under all possible environmental conditions and circumstances. However, Angel's Summer Dream has proven itself to be hardy to United States Department of Agriculture Plant Hardiness Scale Zone eight and higher, which is the equivalent to ten to twenty degrees Fahrenheit and higher. The foliage and stems of Angel's Summer Dream are tender to frost, further emphasizing the tropical nature of all Brugmansia's in general and Angel's Summer Dream in particular.

Certain phenotypical characteristics of Angel's Summer Dream tend to vary based on environmental conditions. For example, the flowers exhibited on the Angel's Summer Dream specimen in the colder winter months tend to be a pale-orange color, while the Angel's Summer Dream will produce blooms of a golden-yellow hue in the warmer conditions of the summer months.

The following traits and characteristics have been observed repeatedly. Due to their distinct nature and differences to traditional Brugmansia characteristics they are determined to be traits unique to Angel's Summer Dream. The combination of all of these characteristics distinguishes Angel's Summer Dream as a unique and distinct Brugmansia cultivar:
1. Angel's Summer Dream specimens exhibit lateral branching, enabling the plant to grow more laterally than vertically
2. Because of the lateral branching of Angel's Summer Dream it is able to flower when it reaches a height of twelve to twenty-four inches
3. Angel's Summer Dream specimens exhibit a longer duration of flowering due to bud formation on axillary spurs along stems.
4. The specimens of the new variety of Brugmansia exhibit early bloom initiation on young plants
5. Angel's Summer Dream has proven to be a compact, free flowering producer of non-white colored flowers.

### BRIEF DISCRIPTION OF THE DRAWINGS

FIG. 1 shows a perspective side view of a specimen of the new variety suspended in a hanging basket.

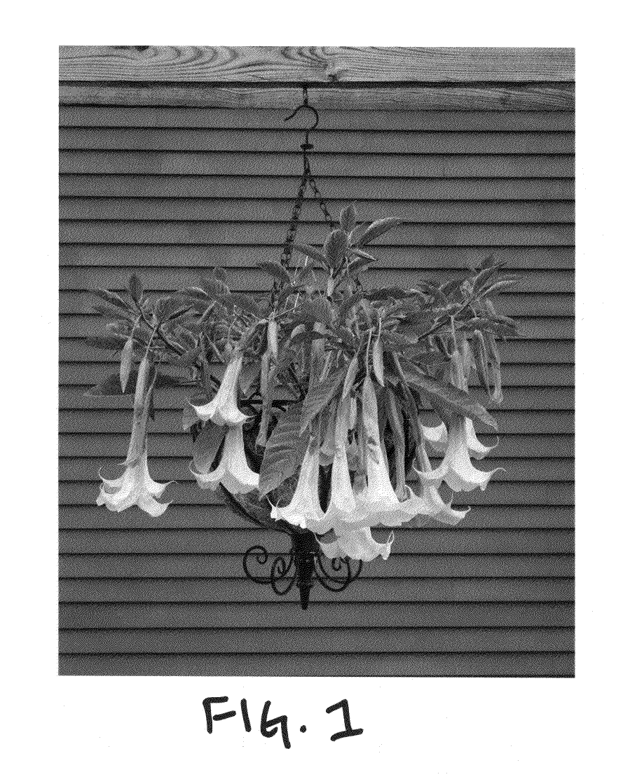

FIG. 2 shows the flower of the plant of the new variety.

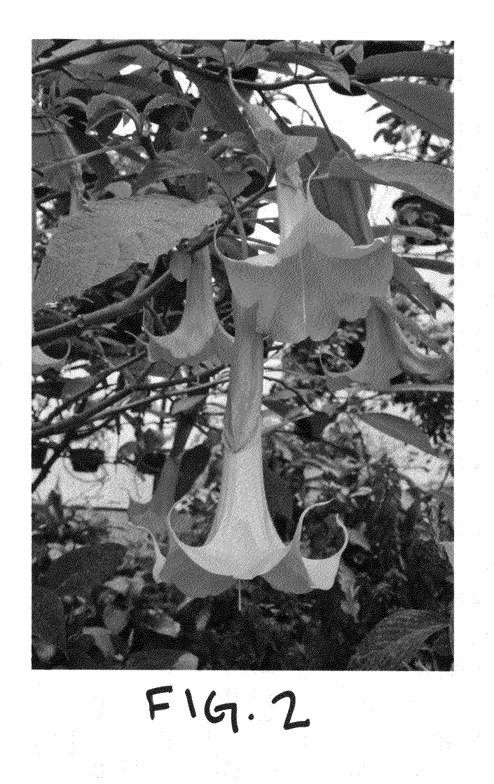

FIG. 3 shows a bottom view of the flowers of the plant of the new variety.

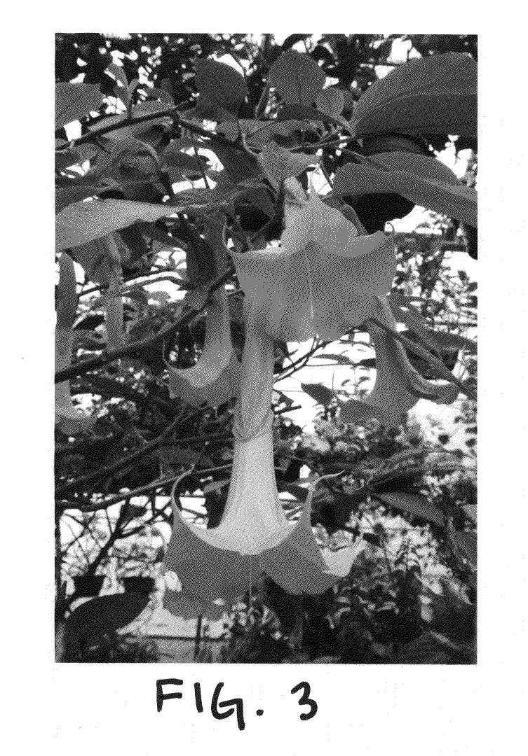

FIG. 4 shows the upper side and the lower side of a mature leaf of the new variety.

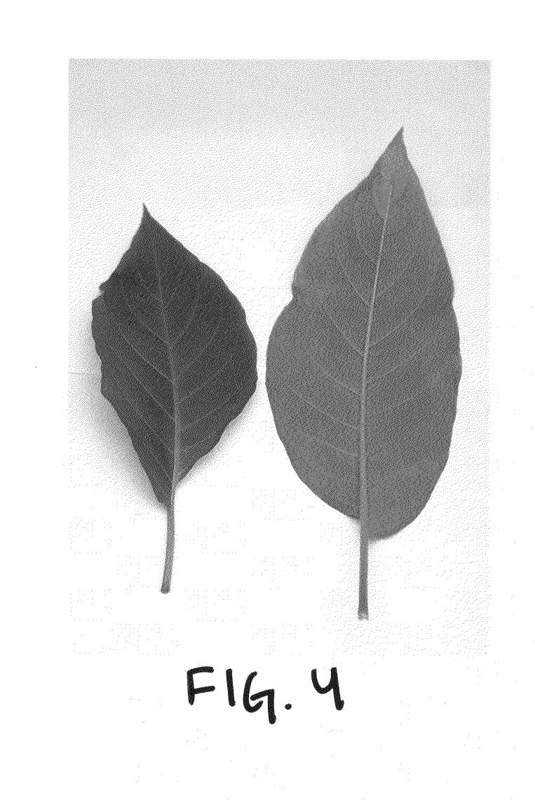

FIG. 5 shows a perspective side view of a typical flowering plant of the new variety.

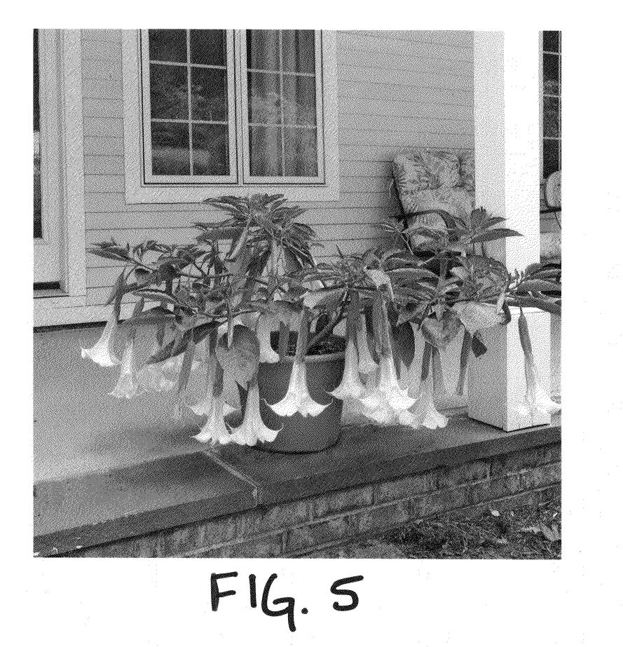

FIG. 6 shows a perspective side view of a typical flower cluster of the plant of the new variety.

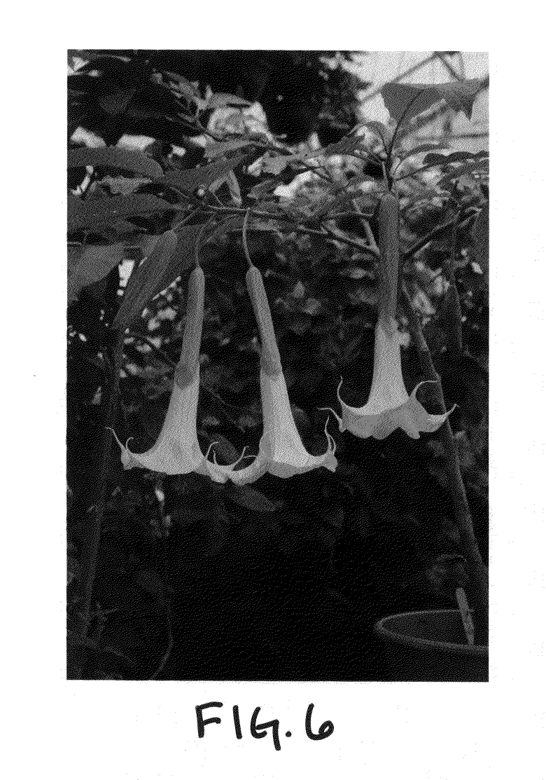

### BOTANICAL DESCRIPTION OF THE PLANT

The aforementioned photographs and following observations exhibit the new variety of plant. Definitive phonological and morphological characteristics of the Brugmansia Angel's Summer Dream are as follows:
Parentage:
   Female, or pod, parent.—Brugmansia hybrida Cherub.
   Male, or seed, parent.—Brugmansia hybrida Inca Sun.
Propagation: Asexual; specifically cutting or tissue culture conducted in Danielson, Conn. in the summer of 2007.
Type: Vegetative cutting or micro propagation
Time to initiate roots: Year round
Time to produce a rooted young plant: Year round
Root description: Fibrous
Appearance: Shrub like habit exhibiting lateral branching
Plant height: 3′-4′
Plant diameter: 3′
Lateral branch description: Length: 2′, diameter: 1′
Foliage description:
   Arrangement.—alternate.
   Shape.—Oblong to elliptic.
   Apex.—Acute.
   Margin.—Smooth.
   Color.—Green.
   Upper surface.—smooth.
   Bottom surface.—Smooth.
   Veins.—Slight pubescent.
Inflorescence description:
   Appearance.—Solitary, trumpet shaped.
   Flowering response.—Flowers year round under direct sunlight.
   Postproduction longevity.—Short lived.
   Quantity of inflorescences per flower stem.—1.
   Length.—10″-12″.
   Diameter.—6″ at its widest.
   Shape.—Trumpet.
   Color.—Orange-yellow.
Peduncles:
   Length.—2.25″.
   Diameter.—0.25″.
   Angle.—Downward.
   Color.—Green.
Reproductive organs:
   Stamen quantity.—6.
   Anther shape.—thin.
   Anther length.—1.25″.
   Anther color.—White.
   Pollen amount.—thin coating.
   Pollen color.—White.
   Pistil quantity.—1.
   Pistil length.—8″.
   Stigma length.— 5/16″.
   Stigma shape.—thin with two opposing groves along the length that are lightly twisted.
Disease/pest resistance: Resistance to pathogens and pests common to Brugmansias has not been observed on plants under commercial greenhouse or outdoor conditions.

I claim:

1. The new and distinct variety of Brugmansia herein described and illustrated and identified by the characteristics enumerated above.
